(19) United States
(12) Patent Application Publication
(10) Pub. No.: US 2010/0288276 A1
(43) Pub. Date: Nov. 18, 2010

(54) COMPOSITIONS FOR TREATING PARKINSON'S DISEASE

(75) Inventors: David Ganderton, Wiltshire (GB); Mark Jonathan Main, Wiltshire (GB); Frazer Giles Morgan, Wiltshire (GB)

Correspondence Address: NIXON PEABODY LLP - PATENT GROUP, 1100 CLINTON SQUARE, ROCHESTER, NY, 14604, US

(73) Assignee: VECTURAL LIMITED, Chippenham, Willshire (GB)

(21) Appl. No.: 12/740,323

(22) PCT Filed: Oct. 31, 2008
(86) PCT No.: PCT/GB2008/003698

§ 371 Date: Jul. 20, 2010

(30) Foreign Application Priority Data
Oct. 31, 2007 (GB) 0721394.5

(51) Int. Cl. A61K 31/4375; A61P 25/16; A61P 25/00; A61M 15/00; A61M 16/10

(52) U.S. Cl. 128/203.15; 514/284; 128/203.16

(57) ABSTRACT

The present invention relates to improved treatment of diseases and disorders of the central nervous system by administration of apomorphine. In particular, the administration is via pulmonary inhalation. The invention provides the means for improving the treatment of a number of conditions, including Parkinson's Disease.

The present invention relates to compositions comprising apomorphine for providing improved treatment of diseases and disorders of the central nervous system, including Parkinson's Disease. In particular, the apomorphine is to be administered via pulmonary inhalation.

### Parkinson's Disease

Parkinson's Disease was first described in England in 1817 by Dr James Parkinson. The disease affects approximately 2 of every 1,000 people and most often develops in those over 50 years of age, affecting both men and women. It is one of the most common neurological disorders of the elderly, and occasionally occurs in younger adults. In some cases, Parkinson's Disease occurs within families, especially when it affects young people. Most of the cases that occur at an older age have no known cause.

The specific of symptoms that an individual experiences vary, but may include tremor of the hands, arms, legs, jaw and face; rigidity or stiffness of the limbs and trunk; bradykinesia or slowness of movement; postural instability or impaired balance and coordination as well as severe depression. Untreated, Parkinson's Disease progresses to total disability, often accompanied by general deterioration of all brain functions, and may lead to an early death.

The symptoms of Parkinson's Disease result from the loss of dopamine-secreting (dopaminergic) cells, in the substantia nigra of the upper part of the brainstem. The exact reason for the wasting of these cells is unknown, although both genetic and environmental factors are known to be important.

There is no known cure for Parkinson's Disease. The goal of treatment is to control symptoms, and medications aim to do this primarily by increasing the levels of dopamine in the brain. The most widely used treatment is L-dopa in various forms. However, this treatment has a number of drawbacks, the most significant being that, due to feedback inhibition, L-dopa results in a reduction in the endogenous formation of L-dopa (and hence dopamine), and so eventually becomes counterproductive. Over time, patients start to develop motor fluctuations, which oscillate between “off” times, a state of decreased mobility, and “on” times, or periods when the medication is working and symptoms are controlled. It is estimated that 40% of Parkinson's patients will experience motor fluctuations within 4-6 years of onset, increasing by 10 percent per year after that.

The average Parkinson's Disease patient experiences 2-3 hours of “off-time” each day. These include handwriting problems, overall slowness, loss of olfaction, loss of energy, stiffness of muscles, walking problems, sleep disturbances, balance difficulties, challenges getting up from a chair, and many other symptoms not related to motor functions, such as sensory symptoms (e.g. pain, fatigue, and motor restlessness); autonomic symptoms (e.g. urinary incontinence and profuse sweats); and psychiatric disorders (e.g. depression, anxiety and psychosis).

One therapeutic approach involves the administration of apomorphine, which is a morphine derivative and dopaminergic agonist. First mooted as a treatment for Parkinson's Disease as early as 1951, the first clinical use of apomorphine was first reported in 1970 by Cotzias et al, although its emetic properties and short half-life made oral use impractical.

The use of apomorphine to treat Parkinson's Disease is effective because of the drug's strong dopaminergic action. However, orally administered apomorphine is associated with an onset period of about 30 to 45 minutes during which the patient suffers unnecessarily. Now, a more common route of administration is by subcutaneous injection. When apomorphine is injected under the skin, it has been shown to bring about an “on” time consistently in 7-10 minutes and to maintain the effect for all areas of fluctuations—motor, sensory and psychiatric—for a period of about 60 minutes.

Whilst apomorphine can be used in combination with L-dopa, the usual intention in the later stages of the disease is to wean patients off L-dopa, as by this stage they will probably be experiencing significant discomfort from off-periods.

Apomorphine has a low incidence of neuropsychiatric problems, and it has thus been used in patients with severe neuropsychiatric complications due to oral anti-Parkinsonian drugs. Injections of apomorphine may help specific symptoms such as off-period pain, belching, screaming, constipation, nocturia, dystonias, erectile impotence, and post-surgical state in selected patients who may not otherwise be candidates for apomorphine.

For subcutaneous administration, the usual dose of apomorphine is 2 mg (provided in a volume of 0.2 ml) per delivery, and it is not recommended to exceed 6 mg in a single off-period because the risk of sensitisation to apomorphine does not outweigh the benefit of the larger doses. The British National Formulary (BNF) recommends that the usual range (after initiation) of a subcutaneous injection is 3 to 30 mg per day to be administered in divided doses. Subcutaneous infusion may be preferable in those patients requiring division of injections into more than 10 doses daily. The maximum single dose is 10 mg, with a total daily dose by either subcutaneous route (or combined routes) that is not to exceed 100 mg.

The recommended continuous subcutaneous infusion dose is initially 1 mg/hour daily and is generally increased according to response (not more often than every 4 hours) in maximum steps of 500 μg/hour, to usual rate of 1 to 4 mg/hour (14 to 60 μg/kg/hour). The infusion site is to be changed every 12 hours and infusion is to be given during waking hours only; 24-hour infusions are not advised unless the patient experiences severe night-time symptoms. Intermittent bolus boosts may also be needed.

However, frequent injection of low doses of apomorphine are often inadequate in controlling the disease symptoms, and in addition to the pain caused by repeated injection, these repeated injections inconvenience the patient, often resulting in non-compliance.

Apomorphine can be administered via subcutaneous infusion using a small pump which is carried by the patient. A low dose is automatically administered throughout the day, reducing the fluctuations of motor symptoms by providing a steady dose of dopaminergic stimulation. However, an additional person (often a spouse or partner) must be responsible for maintenance of the pump, placing a burden on this caregiver.

Of the adverse effects observed with apomorphine administration, nausea and vomiting, and hypotension are the most significant. In light of these adverse effects, the BNF reports that patients are often given anti-emetic prophylaxis three days prior to the initiation of apomorphine therapy and it is recommended that this continue for eight weeks after the apomorphine treatment has finished. Furthermore drowsiness (including sudden onset of sleep), confusion, hallucinations, injection-site reactions (including nodule formation and ulceration), less commonly postural hypotension, breathing difficulties, dyskinesia during “on” periods, haemolytic anaemia with levodopa, rarely eosinophilia, pathological gambling, increased libido and hypersexuality are also reported.

Anti-emetic therapies that may be used include domperidone or trimethobenzamide (trade name Tigan).

The term “parkinsonism” refers to any condition that involves a combination of the types of changes in movement seen in Parkinson's Disease and often has a specific cause, such as the use of certain drugs or frequent exposure to toxic chemicals. Generally, the symptoms of parkinsonism may be treated with the same therapeutic approaches that are applied to Parkinson's Disease.

A dry powder formulation suitable for intranasal delivery of apomorphine is the focus of European Patent No. 0 689 438. The powder formulation comprises particles of apomorphine having a diameter in the range of 50-100 μm in order to avoid accidental pulmonary deposition. Published studies by Britannia Pharmaceuticals Ltd examine the use of nasally administered apomorphine compositions of this kind and have indicated that the onset of pharmaceutical effects is delayed, and the efficacy of these medicaments is reduced in comparison to subcutaneously delivered apomorphine in terms of the percentage decrease in off-period time. Furthermore, some nasal irritation was reported.

Nasal apomorphine formulations have been evaluated by Nastech Inc. for the treatment of Erectile Dysfunction (ED) and Female Sexual Dysfunction (FSD). Although this route of administration presents advantages over the conventional sublingual route of administering apomorphine for treating this condition, intranasal administration does have a number of drawbacks.

The nasal cavity presents a significantly reduced available surface area compared to the lung (1.8 m² versus 200 m²). The nasal cavity is also subjected to natural clearance, which typically occurs every 15-20 minutes, where ciliated cells drive mucus and debris towards the back of the nasopharynx. This action results in a proportion of the apomorphine which is administered to the nose being swallowed, whereupon it is subjected to first-pass metabolism. In contrast, clearance mechanisms in the lung have minimal opportunity to influence absorption as apomorphine rapidly reaches the systemic circulation via transfer across the alveolar membrane.

Challenges to the nasal mucosa, such as congestion or a “bloody” nose will also have a negative impact upon drug absorption following nasal administration. Furthermore, the nasal passage shape and dimension influence drug absorption. Not only are the passages different between patients but there is also a change in shape and dimensions within a patient at different times during the day. Consequently, nasal delivery devices must overcome this significant challenge to ensure reproducible and targeted drug delivery. To ensure delivery to the target site nasal devices typically employ a “forceful” spray which can result in an undesirable sensation. Conversely, inhalers, including dry powder inhalers such as the Vectura's active inhaler device Aspirair® or their passive device Gyrohaler®, produce a patient-friendly drug “cloud” with minimal oral and throat deposition.

Furthermore, extensive literature describes local apomorphine-attributed irritation following intranasal administration with a number of patients reporting episodes of severe or disabling nasal complications including irritation, crusting, blockage, bleeding, burning immediately after dosing and vestibulitis leading to premature discontinuation of study treatment.

Nevertheless, the apomorphine nasal powder developed by Britannia Pharmaceuticals is said to offer a rapid onset that is comparable to subcutaneous injection and much faster than oral dosing, as well as bioavailability that is also comparable to the subcutaneous route of administration.

It has now been discovered that the delivery of apomorphine by pulmonary inhalation provides increased delivery efficiency, increased bioavailability and consistent absorption with an ultimately faster and more predictable clinical effect compared to other routes of administration.

U.S. Pat. No. 6,193,954 (Abbott Laboratories) relates to formulations for pulmonary delivery of dopamine agonists. The dopamine agonist is in the form of a microparticle or powder and is to be delivered to the lung dispersed in a liquid vehicle.

U.S. Pat. No. 6,514,482 (Advanced Inhalation Research, Inc.) claims a method of providing “rescue therapy” in the treatment of Parkinson's Disease in which particles of apomorphine are delivered to the pulmonary system. Rescue therapy normally refers to non-surgical medical treatment in life-threatening situations. However, despite the unpleasantness of Parkinson's Disease, the symptoms are not life threatening and this patent would therefore appear to relate to “rescue” from off-period symptoms. As used within U.S. Pat. No. 6,514,482, “rescue therapy” means on-demand, rapid delivery of a drug to a patient to help reduce or control disease symptoms.

In the prior art, the dopamine agonist compositions and the methods of treating Parkinson's Disease involve administering fixed doses of apomorphine at the onset of off-period symptoms. This does not provide the optimal treatment. It would be highly beneficial to be able to readily determine the appropriate dose of apomorphine to suit the specific needs of an individual patient. This would ensure that the minimum necessary dose is, administered. Such a self-titrating system should be flexible, to enable the dose to be tailored to the patient without the need for different strength presentations. The system should also allow the self-titrating to be on-going, with the patient able to constantly change the dose of apomorphine to meet his or her symptoms and needs. This is desirable for a number of reasons, not least in order to minimise the adverse side effects associated with the treatment (including emesis) and to reduce the risk of apomorphine sensitisation.

It is a further aim to reduce “off-periods” experienced by the patient as much as possible and, if possible, to avoid such off-periods altogether. It is desirable to achieve this without the need to administer excessively large doses of apomorphine (especially in terms of the daily dose administered to the patient over a 24-hour period).

It is also clearly desirable to provide a composition or treatment regimen which the patient is able to self-administer, reducing the burden on the care-giver. A safe and convenient, pain-free route of administration is clearly preferable to constant and frequent injections or a permanent infusion pump. A medication which alleviates this dependency while allowing ease of delivery for frequent administration of apomorphine would clearly be an advantage.

A formulation that is capable of maintaining an extended duration of response would provide the patient with a window in which they could self administer the next dose, thereby negate the need for caregiver assistance.

A method of administration which reduces the emetic effects of apomorphine would obviously be advantageous.

It is also desirable to provide apomorphine compositions which are stable over time under normal storage conditions, in order to avoid the significant expense associated with the disposal of spoiled medicine.

In particular, therefore, there is a need for a composition comprising apomorphine in a stable, dry powder form suitable for the straightforward administration of low doses of drug with a sufficiently low induction of emesis and rapid onset of pharmacological effects to facilitate self-titration and optimisation of levels of medication.

Nasal administration of apomorphine results in a Tₘₐₓ of approximately 15 minutes. Pulmonary administration results in a Tₘₐₓ of less than 1 minute in some patients. This is thought to be equivalent to the Tₘₐₓ observed following subcutaneous administration. Pulmonary administration has greater bioavailability than nasal administration. This, in turn, means that nasal doses need to be increased in order to compensate for the lower bioavailability.

In the Apokyn® information sheet dated April 2004, it is stated that apomorphine hydrochloride is a lipophilic compound that is rapidly absorbed (time to peak concentration ranges from 10 to 60 minutes) following subcutaneous administration into the abdominal wall. After subcutaneous administration, apomorphine appears to have bioavailability equal to that of an intravenous administration. Apomorphine exhibits linear pharmacokinetics over a dose range of 2 to 8 mg following a single subcutaneous injection of apomorphine into the abdominal wall in patients with idiopathic Parkinson's disease.

Based upon the assertion that the bioavailability of subcutaneously administered apomorphine is equal to that of intravenously administered apomorphine, it is surprising that the bioavailability of apomorphine administered by pulmonary inhalation is comparable, if not greater than the bioavailability following subcutaneous injection. This is most unexpected.

### SUMMARY OF THE INVENTION

In a first aspect of the present invention, a dry powder composition comprising apomorphine for administration by pulmonary inhalation is provided, for treating conditions of the central nervous system, including Parkinson's Disease (PD).

The combination of lung pathophysiology and inhaled apomorphine attributes result in rapid and consistent systemic exposure which translates into a rapid and predictable therapeutic effect, both of which are key requirements when considering improved treatments of PD. Preferably, a Tₘₐₓ of as little as 1 minute is observed. The majority of patients achieved conversions (that is, the onset of the therapeutic effect) within 10 minutes of inhaling apomorphine. Some patients reported conversion from the “off” to the “on” state as quickly as 2 minutes after administration of the apomorphine by pulmonary inhalation.

In one embodiment, the composition comprises a dose of apomorphine to be administered to a patient, the amount of apomorphine being up to 15 mg, 14 mg, 13 mg, 12 mg, 11 mg, 10 mg, 9 mg, 8 mg, 7 mg, 6 mg or up to 5 mg. Preferably the dose is at least 1 mg, 2 mg, 3 mg or 4 mg. The dose may be a figure comprised within a range defined by any of the lower dose values with any of the higher dose values, for example at least 1 mg and up to 15 mg, at least 2 mg and up to 15 mg, at least 3 mg and up to 15 mg, at least 1 mg and up to 14 mg, at least 1 mg and up to 13 mg, and so on.

In one aspect the dose is a Nominal dose. The Nominal Dose (ND) is the amount of drug metered in the receptacle (also known as the Metered Dose). This is different to the amount of drug that is delivered to the patient which is referred to a Delivered Dose.

The fine particle fraction (FPF) is normally defined as the FPD (the dose that is <5 μm) divided by the Emitted Dose (ED) which is the dose that leaves the device. The FPF is expressed as a percentage. Herein, the FPF of ED is referred to as FPF (ED) and is calculated as FPF (ED)=(FPD/ED)×100%.

The fine particle fraction (FPF) may also be defined as the FPD divided by the Metered Dose (MD) which is the dose in the blister or capsule, and expressed as a percentage. Herein, the FPF of MD is referred to as FPF (MD), and is calculated as FPF (MD)=(FPD/MD)×100%.

In a preferred embodiment, the dose is administered to the patient as a single dose requiring just one inhalation. In one embodiment, the dose is preferably provided in a blister or capsule which is to be dispensed using a dry powder inhaler device. Alternatively, the dose may be dispensed using a pressurised metered dose inhaler (pMDI). Typically, administration of a dose of the compositions according to the present invention will result in a fine particle dose (FPD) of about 2 to about 6 mg, and preferably of about 4 mg of apomorphine. These doses are administered to the pulmonary mucosa and the apomorphine is absorbed.

In yet another embodiment, the doses of the apomorphine composition are to be administered to the patient as needed, that is, when the patient experiences or suspects the onset of an off-period. This provides an “on-demand” treatment. In this embodiment, a single effective dose of apomorphine may be administered. Alternatively, multiple smaller doses may be administered sequentially, with the effect of each dosing being assessed by the patient before the next dose is administered. This allows self-titration and optimisation of the dose.

In another embodiment, the composition provides a daily dose, which is the dose administered over a period of 24 hours, of between about 30 and about 110 mg. The daily does will often be divided up into a number of doses. Preferably, the daily dose is between about 50 and about 80 mg. These daily doses may be administered at a single instance (usually involving multiple inhalations), but it is expected that the daily dose will be spread out over the 24 hour period with patients receiving, on average, 2-3 separate single administrations, although some patients may receive 5-6 doses, with a daily extreme of 10 doses of 11 mg per dose, i.e. 110 mg in a 24 hour period. It is important to note that the dose recommendations vary depending on medical authority with a single dose of 10 mg and 6 mg and a maximum daily dose of 100 mg and approximately 25 mg being recommended in Europe and the United States of America respectively.

In another embodiment, the composition allows doses to be administered at regular and frequent intervals, for example intervals of about 60 minutes, about 45 minutes, about 30 minutes, about 20 minutes, about 15 minutes or about 10 minutes, providing maintenance therapy to avoid the patient experiencing off-periods comparable to the effect of the infusion pump mentioned above. In such an embodiment, the individual doses administered at the chosen intervals will be adjusted to provide a daily dose within safe limits, whilst hopefully providing the patient with adequate relief from symptoms. For example, each individual fine particle dose would preferably provide in the order of about 0.5 mg to about 7 mg apomorphine, more preferably 2 mg to 6 mg, more preferably 3 mg to 5 mg, and most preferably about 4.5 mg. A fine particle dose within this range will be possible from nominal dose of about 0.8 mg to 11.5 mg, 3 mg to 10 mg and about 7 mg respectively. In one aspect each individual fine particle dose would provide in the order of about 0.5 mg to about 3 mg apomorphine, and in one aspect would provide about 1.6 mg. If the dosing takes place over a period of 11.5 hours (when the patient is awake) and at 10 minute intervals, this will provide a daily dose of 110 mg.

According to one embodiment of the present invention, a composition comprising apomorphine is provided, wherein the administration of the composition by pulmonary inhalation provides a Cₘₐₓ within less than about 10 minutes and preferably within about 5 minutes of administration, with about 2 minutes of administration or even within 1 minute of administration. Preferably, the Cₘₐₓ is provided within 1 to 5 minutes.

In a further embodiment of the present invention, the administration of the composition by pulmonary inhalation provides a dose dependent Cₘₐₓ.

In accordance with another embodiment of the present invention, a dose of apomorphine is inhaled into the lungs and said dose is sufficient to provide a therapeutic effect in about 10 minutes or less. In some cases, the therapeutic effect is experienced within as little as about 5 minutes, about 2 minutes or even about 1 minute from administration.

In another embodiment of the invention, the administration of the composition by pulmonary inhalation provides a terminal elimination half-life of between 30 and 70 minutes.

In yet another embodiment, the administration of the composition by pulmonary inhalation provides a therapeutic effect with a duration of at least 45 minutes, preferably at least 60 minutes. In a clinical trial, a mean duration of the therapeutic effect of 75 minutes was observed.

In a further embodiment, the composition comprises at least about 70% (by weight) apomorphine, or at least about 75%, 80%, 85%, 90%, 95%, 96%, 97%, 98% or 99% (by weight) apomorphine.

In a yet further embodiment, the compositions according to the present invention are for use in providing treatment of the symptoms of Parkinson's Disease or for preventing the symptoms altogether. The patient is preferably able to administer a dose and to ascertain within a period of no more than about 10 minutes whether that administered dose is sufficient to treat or prevent the symptoms of Parkinson's Disease. If a further dose is felt to be necessary, this may be safely administered and the procedure may be repeated until the desired therapeutic effect is achieved.

This self-titration of the apomorphine dose is possible as a result of the rapid onset of the therapeutic effect, the accurate and relatively small dose of apomorphine and the low incidence of side effects, including emesis. It is also important that the mode of administration is painless and convenient, allowing repeated dosing without undue discomfort or inconvenience.

According to a second aspect of the present invention, blisters, capsules, reservoir dispensing systems and the like are provided, comprising doses of the compositions according to the first aspect of the invention.

According to a third aspect of the present invention, inhaler devices are provided for dispensing doses of the compositions according to the first aspect of the invention. In one embodiment of the present invention, the inhalable compositions are administered via a dry powder inhaler (DPI). In an alternative embodiment, the compositions are administered via a pressurized metered dose inhaler (pMDI), or via a nebulised system.

According to a fourth aspect of the present invention, processes are provided for preparing the compositions according to the first aspect of the invention.

According to a fifth aspect of the present invention, methods of treating diseases of the central nervous system, such as Parkinson's Disease are provided, the treatment involving administering doses of the compositions according to the first aspect of the invention by pulmonary inhalation.

Alternatively, the use of apomorphine in the manufacture of a medicament for treating diseases of the central nervous system, such as Parkinson's Disease is provided, wherein the apomorphine is to be administered by pulmonary inhalation. In a preferred embodiment, the apomorphine is in the form of a composition according to the first aspect of the present invention.

New methods of treating diseases of the central nervous system, such as Parkinson's Disease are provided, using new pharmaceutical compositions comprising apomorphine which are administered by pulmonary inhalation. These methods achieve the desired therapeutic effect whilst avoiding the side effects associated with the administration of apomorphine, especially when apomorphine is administered in the relatively large doses usually associated with treating conditions such as Parkinson's Disease.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### DETAILED DESCRIPTION OF THE INVENTION

The present invention relates to high performance inhaled delivery of apomorphine, which has a number of significant and unexpected advantages over previously used modes of administration. The mode of administration and the compositions of the present invention make this excellent performance possible. However, it is important that the apomorphine is delivered in such a way that will allow rapid absorption of an accurate and consistent amount of apomorphine to provide a predictable therapeutic effect. This is made more difficult because of the relatively large amount of drug that must be administered.

The advantages for this pulmonary route of administration are improved safety, reduced exposure variability resulting in reduced incidence of dyskinesia, more rapid onset of action compared to subcutaneous and a non-invasive route of administration.

### Apomorphine Compositions for Pulmonary Inhalation

Since the effective treatment of PD requires the delivery of a relatively large dose of apomorphine there are significant technical hurdles to overcome. To date, dry powder inhaler devices have tended to deliver doses of up to 3 mg of powder or occasionally up to 20 mg. Doses delivered by pressurised metered dose inhalers are of the order of 1 μg to 3 mg. In contrast, it is intended to provide a dose of some 11 mg of a dry powder composition comprising apomorphine in a single inhalation in order to provide an effective and user-friendly treatment of PD. The volume (dose) of the dry powder formulations according to the invention to be administered by inhalation may be as high as 40 mg, and in one aspect may be as high as 50 mg. When the dose of powder composition is so large, it is envisaged that the Nominal Dose will be in the region of 7 mg and the FPD approximately 4 mg.

In the past, many of the commercially available dry powder inhalers exhibited very poor dosing efficiency, with sometimes as little as 10% of the active agent present in the dose actually being properly delivered to the user so that it can have a therapeutic effect. This low efficiency is simply not acceptable where a high dose of active agent is required for the desired therapeutic effect.

The reason for the lack of dosing efficiency is that a proportion of the active agent in the dose of dry powder tends to be effectively lost at every stage the powder goes through from expulsion from the delivery device to deposition in the lung. For example, substantial amounts of material may remain in the blister/capsule or device. Material may be lost in the throat of the subject due to excessive plume velocity. However, it is frequently the case that a high percentage of the dose delivered exists in particulate forms of aerodynamic diameter in excess of that required.

It is well known that particle impaction in the upper airways of a subject is predicted by the so-called impaction parameter. The impaction parameter is defined as the velocity of the particle multiplied by the square of its aerodynamic diameter. Consequently, the probability associated with delivery of a particle through the upper airways region to the target site of action, is related to the square of its aerodynamic diameter. Therefore, delivery to the lower airways, or the deep lung is dependant on the square of its aerodynamic diameter, and smaller aerosol particles are very much more likely to reach the target site of administration in the user and therefore able to have the desired therapeutic effect.

Particles having aerodynamic diameters of less than 10 μm tend to be deposited in the lung. Particles with an aerodynamic diameter in the range of 2 μm to 5 μm will generally be deposited in the respiratory bronchioles whereas smaller particles having aerodynamic diameters in the range of 0.05 to 3 μm are likely to be deposited in the alveoli. So, for example, high dose efficiency for particles targeted at the alveoli is predicted by the dose of particles below 3 μm, with the smaller particles being most likely to reach that target site.

In one embodiment of the present invention, the composition comprises active particles comprising apomorphine, at least 50%, at least 70% or at least 90% of the active particles having a Mass Median Aerodynamic Diameter (MMAD) of no more than about 10 μm. In another embodiment, at least 50%, at least 70% or at least 90% of the active particles have an MMAD of from about 2 μm to about 5 μm. In yet another embodiment, at least 50%, at least 70% or at least 90% of the active particles have aerodynamic diameters in the range of about 0.05 μm to about 3 μm. In one embodiment of the invention, at least about 90% of the particles of apomorphine have a particle size of 5 μm or less.

Particles having a diameter of less than about 10 μm are, however, thermodynamically unstable due to their high surface area to volume ratio, which provides significant excess surface free energy and encourages particles to agglomerate. In a dry powder inhaler, agglomeration of small particles and adherence of particles to the walls of the inhaler are problems that result in the active particles leaving the inhaler as large agglomerates or being unable to leave the inhaler and remaining adhered to the interior of the device, or even clogging or blocking the inhaler.

The uncertainty as to the extent of formation of stable agglomerates of the particles between each actuation of the inhaler, and also between different inhalers and different batches of particles, leads to poor dose reproducibility. Furthermore, the formation of agglomerates means that the MMAD of the active particles can be vastly increased, with agglomerates of the active particles not reaching the required part of the lung. Consequently, it is essential for the present invention to provide a powder formulation which provides good dosing efficiency and reproducibility, delivering an accurate and predictable dose.

Much work has been done to improve the dosing efficiency of dry powder systems comprising active particles having a size of less than 10 μm; reducing the loss of the pharmaceutically active agent at each stage of the delivery. In the past, efforts to increase dosing efficiency and to obtain greater dosing reproducibility have tended to focus on preventing the formation of agglomerates of fine particles of active agent. Such agglomerates increase the effective size of these particles and therefore prevent them from reaching the lower respiratory tract or deep lung, where the active particles should be deposited in order to have their desired therapeutic effect. Proposed measures have included the use of relatively large carrier particles. The fine particles of active agent tend to become attached to the surfaces of the carrier particles as a result of interparticle forces such as Van der Waals forces. Upon actuation of the inhaler device, the active particles are supposed to detach from the carrier particles and are then present in the aerosol cloud in inhalable form. In addition or as an alternative, the inclusion of additive materials that act as force control agents that modify the cohesion and adhesion between particles has been proposed.

However, where the dose of drug to be delivered is very high, the options for adding materials to the powder composition are limited, especially where at least 70% of the compositions is made up of the apomorphine as is preferred in the present invention. Nevertheless, it is imperative that the dry powder composition exhibit good flow and dispersion properties, to ensure good dosing efficiency.

The term “ultrafine particle dose” (UFPD) is used herein to mean the total mass of active material delivered by a device which has a diameter of not more than 3 μm. The term “ultrafine particle fraction” is used herein to mean the percentage of the total amount of active material delivered by a device which has a diameter of not more than 3 μm. The term percent ultrafine particle dose (% UFPD) is used herein to mean the percentage of the total metered dose which is delivered with a diameter of not more than 3 μm (i.e., % UFPD=100*UFPD/total metered dose).

The terms “delivered dose” and “emitted dose” or “ED” are used interchangeably herein. These are measured as set out in the current EP monograph for inhalation products.

“Actuation of an inhaler” refers to the process during which a dose of the powder is removed from its rest position in the inhaler. That step takes place after the powder has been loaded into the inhaler ready for use.

In one embodiment of the present invention, the composition used for treating conditions of the central nervous system, including Parkinson's Disease via inhalation comprises a dose of from about 1.5 mg FPD of apomorphine (that is, apomorphine, apomorphine free base, pharmaceutically acceptable salt(s) or ester(s) thereof, based on the weight of the hydrochloride salt). The dose may comprise from about 100 to 1500 μg FPD of said apomorphine.

In another embodiment of the present invention, the composition used for treating conditions of the central nervous system, including Parkinson's Disease via inhalation comprises a nominal dose of from about 4 mg of apomorphine (that is, apomorphine, apomorphine free base, pharmaceutically acceptable salt(s) or ester(s) thereof, based on the weight of the hydrochloride salt) said dose may achieve from about 1.5-3.5 mg FPD of said apomorphine, such as 2.5-3.5 mg FPD when delivered from a passive dry powder inhaler.

In another embodiment of the present invention, the dose of the powder composition delivers, in vitro, a fine particle dose of from about 400 μg to about 6000 μg of apomorphine, such as from about 400 μg to about 4000 μg of apomorphine(based on the weight of the hydrochloride salt), when measured by a Multistage Liquid Impinger, United States Pharmacopoeia 26, Chapter 601, Apparatus 4 (2003), an Andersen Cascade Impactor or a New Generation Impactor. Preferably, the dose delivers, in vitro, a fine particle dose from about 400 to about 5000 μg, and in one aspect a fine particle dose from about 400 to about 4000 μg of apomorphine.

In the context of the present invention, the dose (e.g., in micrograms) of apomorphine or its pharmaceutically acceptable salts or esters will be described based upon the weight of the hydrochloride salt (apomorphine hydrochloride).

The tendency of fine particles to agglomerate means that the FPF of a given dose can be highly unpredictable and a variable proportion of the fine particles will be administered to the lung, or to the correct part of the lung, as a result. This is observed, for example, in formulations comprising pure drug in fine particle form. Such formulations exhibit poor flow properties and poor FPF.

In an attempt to improve this situation and to provide a consistent FPF and FPD, dry powder compositions according to the present invention may include additive material which is an anti-adherent material and reduces cohesion between the particles in the composition.

The additive material is selected to reduce the cohesion between particles in the dry powder composition. It is thought that the additive material interferes with the weak bonding forces between the small particles, helping to keep the particles separated and seducing the adhesion of such particles to one another, to other particles in the formulation if present and to the internal surfaces of the inhaler device. Where agglomerates of particles are formed, the addition of particles of additive material decreases the stability of those agglomerates so that they are more likely to break up in the turbulent air stream created on actuation of the inhaler device, whereupon the particles are expelled from the device and inhaled. As the agglomerates break up, the active particles may return to the form of small individual particles or agglomerates of small numbers of particles which are capable of reaching the lower lung.

The additive material may be in the form of particles which tend to adhere to the surfaces of the active particles, as disclosed in WO 1997/03649. Alternatively, the additive material may be coated on the surface of the active particles by, for example a co-milling method as disclosed in WO 2002/43701.

Preferably, the additive material is an anti-adherent material and it will tend to reduce the cohesion between particles and will also prevent fine particles becoming attached to surfaces within the inhaler device. Advantageously, the additive material is an anti-friction agent or glidant and will give the powder formulation better flow properties in the inhaler. The additive materials used in this way may not necessarily be usually referred to as anti-adherents or anti-friction agents, but they will have the effect of decreasing the cohesion between the particles or improving the flow of the powder. The additive materials are sometimes referred to as force control agents (FCAs) and they usually lead to better dose reproducibility and higher FPFs.

Therefore, an additive material or FCA, as used herein, is a material whose presence on the surface of a particle can modify the adhesive and cohesive surface forces experienced by that particle, in the presence of other particles and in relation to the surfaces that the particles are exposed to. In general, its function is to reduce both the adhesive and cohesive forces.

The reduced tendency of the particles to bond strongly, either to each other or to the device itself, not only reduces powder cohesion and adhesion, but can also promote better flow characteristics. This leads to improvements in the dose reproducibility because it reduces the variation in the amount of powder metered out for each dose and improves the release of the powder from the device. It also increases the likelihood that the active material, which does leave the device, will reach the lower lung of the patient.

It is favourable for unstable agglomerates of particles to be present in the powder when it is in the inhaler device. As indicated above, for a powder to leave an inhaler device efficiently and reproducibly, the particles of such a powder should be large, preferably larger than about 40 μm. Such a powder may be in the form of either individual particles having a size of about 40 μm or larger and/or agglomerates of finer particles, the agglomerates having a size of about 40 μm or larger. The agglomerates formed can have a size of 100 μm or 200 μm and, depending on the type of device used to dispense the formulation, the agglomerates may be as much as about 1000 μm. With the addition of the additive material, those agglomerates are more likely to be broken down efficiently in the turbulent airstream created on inhalation. Therefore, the formation of unstable or “soft” agglomerates of particles in the powder may be favoured compared with a powder in which there is substantially no agglomeration. Such unstable agglomerates are stable whilst the powder is inside the device but are then disrupted and broken up upon inhalation.

It is particularly advantageous for the additive material to comprise an amino acid. Amino acids have been found to give, when present as additive material, high respirable fraction of the active material and also good flow properties of the powder. A preferred amino acid is leucine, in particular L-leucine, di-leucine and tri-leucine. Although the L-form of the amino acids is generally preferred, the D- and DL-forms may also be used. The additive material may comprise one or more of any of the following amino acids: aspartame, leucine, isoleucine, lysine, valine, methionine, cysteine, and phenylalanine. Additive materials may also include, for example, metal stearates such as magnesium stearate, phospholipids, lecithin, colloidal silicon dioxide and sodium stearyl fumarate, and are described more fully in WO 1996/23485, which is hereby incorporated by reference.

Advantageously, the powder includes at least 80%, preferably at least 90% by weight of apomorphine (or its pharmaceutically acceptable salts) based on the weight of the powder. The optimum amount of additive material will depend upon the precise nature of the additive and the manner in which it is incorporated into the composition. In some embodiments, the powder advantageously includes not more than 8%, more advantageously not more than 5% by weight of additive material based on the weight of the powder. As indicated above, in some cases it will be advantageous for the powder to contain about 1% by weight of additive material. In other embodiments, the additive material or FCA may be provided in an amount from about 0.1% to about 10% by weight, and preferably from about 0.15% to 5%, most preferably from about 0.5% to about 2%.

When the additive material is micronised leucine or lecithin, it is preferably provided in an amount from about 0.1% to about 10% by weight. Preferably, the additive material comprises from about 3% to about 7%, preferably about 5%, of micronised leucine. Preferably, at least 95% by weight of the micronised leucine has a particle diameter of less than 150 μm, preferably less than 100 μm, and most preferably less than 50 μm. Preferably, the mass median diameter of the micronised leucine is less than 10 μm.

If magnesium stearate or sodium stearyl fumarate is used as the additive material, it is preferably provided in an amount from about 0.05% to about 10%, from about 0.15% to about 5%, from about 0.25% to about 3%, or from about 0.5% to about 2.0% depending on the required final dose.

In a further attempt to improve extraction of the dry powder from the dispensing device and to provide a consistent FPF and FPD, dry powder compositions according to the present invention may include particles of an inert excipient material, which act as carrier particles. These carrier particles are mixed with fine particles of active material and any additive material which is present. Rather than sticking to one another, the fine active particles tend to adhere to the surfaces of the carrier particles whilst in the inhaler device, but are supposed to release and become dispersed upon actuation of the dispensing device and inhalation into the respiratory tract, to give a fine suspension.

The inclusion of carrier particles is less attractive where very large doses of active agent are to be delivered, as they tend to significantly increase the volume of the powder composition. Nevertheless, in some embodiments of the present invention, the compositions include carrier particles. In such an embodiment, the composition comprises at least about 10% (by weight) apomorphine, or at least about 15%, 17%, or 18% or 18.5% (by weight) apomorphine. Preferably, the carrier particles are present in small amount, such as no more than 90%, preferably 85%, more preferably 83%, more preferably 80% by weight of the total composition, in which the total apomorphine and magnesium stearate content would be about 18.5% and 1.5% by weight respectively.

Carrier particles may be of any acceptable inert excipient material or combination of materials. For example, the carrier particles may be composed of one or more materials selected from sugar alcohols, polyols and crystalline sugars. Other suitable carriers include inorganic salts such as sodium chloride and calcium carbonate, organic salts such as sodium lactate and other organic compounds such as polysaccharides and oligosaccharides. Advantageously, the carrier particles comprise a polyol. In particular, the carrier particles may be particles of crystalline sugar, for example mannitol, trehalose, melezitose, dextrose or lactose. Preferably, the carrier particles are composed of lactose.

Thus, in one embodiment of the present invention, the composition comprises active particles comprising apomorphine and carrier particles. The carrier particles may have an average particle size of from about 5 to about 1000 μm, from about 4 to about 40 μm, from about 60 to about 200 μm, or from 150 to about 1000 μm. Other useful average particle sizes for carrier particles are about 20 to about 30 μm or from about 40 to about 70 μm.

In an alternate embodiment, the carrier particles are present in small amount, such as no more than 80%, preferably no more than 70%, more preferably no more than 60%, more preferably no more than 50% by weight of the total composition. Where the carrier is present in an amount of 80% then in one aspect the total apomorphine and magnesium stearate content would be 18% and 2% by weight respectively. As the amount of carrier in these formulations changes, the amounts of additive and apomorphine will also change, but the ratio of these constituents preferably remains approximately 1:9 to about 1:13.

In an alternate embodiment, the formulation does not contain carrier particles and comprises apomorphine and additive, such as at least 30%, preferably 60%, more preferably 80%, more preferably 90% more preferably 95% and most preferably 97% by weight of the total composition comprises of pharmaceutically active agent. The active agent may be apomorphine alone or it may be a combination of the apomorphine and an anti-emetic drug or another drug which would benefit Parkinson's Disease patients. The remaining components may comprise one or more additive materials, such as those discussed above.

In a further embodiment the formulation may contain carrier particles and comprises apomorphine and additive, such as at least 30%, preferably 60%, more preferably 80%, more preferably 90% more preferably 95% and most preferably 97% by weight of the total composition comprises the pharmaceutically active agent and wherein the remaining components comprise additive material and larger particles. The larger particles provide the dual action of acting as a carrier and facilitating powder flow.

In a preferred embodiment, the composition comprises apomorphine (30% w/w) and lactose having an average particles size of 45-65 μm.

The compositions comprising apomorphine and carrier particles may further include one or more additive materials. The additive material may be in the form of particles which tend to adhere to the surfaces of the active particles, as disclosed in WO 1997/03649. Alternatively, the additive material may be coated on the surface of the active particles by, for example a co-milling method as disclosed in WO 2002/43701 or on the surfaces of the carrier particles, as disclosed in WO 2002/00197.

In one embodiment, the additive is coated onto the surface of the carrier particles. This coating may be in the form of particles of additive material adhering to the surfaces of the carrier particles (by virtue of interparticle forces such as Van der Waals forces), as a result of a blending of the carrier and additive. Alternatively, the additive material may be smeared over and fused to the surfaces of the carrier particles, thereby forming composite particles with a core of inert carrier material and additive material on the surface. For example, such fusion of the additive material to the carrier particles may be achieved by co-jet milling particles of additive material and carrier particles. In some embodiments, all three components of the powder (active, carrier and additive) are processed together so that the additive becomes attached to or fused to both the carrier particles and the active particles. In one illustrative embodiment, the compositions include an additive material, such as magnesium stearate (up to 10% w/w) or leucine, said additive being jet-milled with the particles of apomorphine and/or with the lactose.

In certain embodiments of the present invention, the apomorphine formulation is a “carrier free” formulation, which includes only the apomorphine or its pharmaceutically acceptable salts or esters and one or more additive materials.

Advantageously, in these “carrier free” formulations, at least 90% by weight of the particles of the powder have a particle size less than 63 μm, preferably less than 30 μm and more preferably less than 10 μm. As indicated above, the size of the apomorphine (or its pharmaceutically acceptable salts) particles of the powder should be within the range of about from 0.1 μm to 5 μm for effective delivery to the lower lung. Where the additive material is in particulate form, it may be advantageous for these additive particles to have a size outside the preferred range for delivery to the lower lung.

The powder includes at least 60% by weight of the apomorphine or a pharmaceutically acceptable salt or ester thereof based on the weight of the powder. Advantageously, the powder comprises at least 70%, or at least 80% by weight of apomorphine or a pharmaceutically acceptable salt or ester thereof based on the weight of the powder. Most advantageously, the powder comprises at least 90%, at least 95%, or at least 97% by weight of apomorphine or a pharmaceutically acceptable salt or ester thereof based on the weight of the powder. It is believed that there are physiological benefits in introducing as little powder as possible to the lungs, in particular material other than the active ingredient to be administered to the patient. Therefore, the quantities in which the additive material is added are preferably as small as possible. In one aspect the powder, therefore, would comprise more than 99% by weight of apomorphine or a pharmaceutically acceptable salt or ester thereof.

Apomorphine can exist in a free base form or as an acid addition salt. For the purposes of the present invention apomorphine hydrochloride and the apomorphine free base forms are preferred, but other pharmacologically acceptable forms of apomorphine can also be used. The term “apomorphine” as used herein includes the free base form of this compound as well as the pharmacologically acceptable salts or esters thereof. In a preferred embodiment, at least some of the apomorphine is in amorphous form. A formulation containing amorphous apomorphine will possess preferable dissolution characteristics. A stable form of amorphous apomorphine may be prepared using suitable sugars such as trehalose and melezitose.

In addition to the hydrochloride salt, other acceptable acid addition salts include the hydrobromide, the hydroiodide, the bisulfate, the phosphate, the acid phosphate, the lactate, the citrate, the tartrate, the salicylate, the succinate, the maleate, the gluconate, and the like.

As used herein, the term “pharmaceutically acceptable esters” of apomorphine refers to esters formed with one or both of the hydroxyl functions at positions 10 and 11, and which hydrolyse in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butryates, acrylates and ethyl succinates.

The free base of apomorphine is particularly attractive in the context of the present invention as it crosses the lung barrier very readily and so it is anticipated that its administration via pulmonary inhalation will exhibit extremely fast onset of the therapeutic effect. Thus, any of the compositions disclosed herein may be formulated using the apomorphine free base. Alternatively, apomorphine hydrochloride hemi-hydrate is also a preferred form.

### Pharmacokinetics

The concept of bioavailability within the desired time period is of therapeutic interest is paramount importance when avoiding “off” periods. When this is achieved, rapid therapeutic relief is ensured.

In one embodiment of the present invention, a Nominal Dose includes about 400 to about 1600 μg of apomorphine hydrochloride, and the dose provides, in vivo, a mean Cₘₐₓ of from about 3.03±0.71 ng/ml to about 11.92±1.17 ng/ml. The Cₘₐₓ for any dose of apomorphine occurs between 1 and 30 minutes after administration pulmonary inhalation, and preferably after between 0.1 and 5 minutes and most preferably between 0.1 and 2 minutes. The terminal elimination of the drug is approximately one hour for any dose. The elimination half life for a dose of apomorphine delivered by pulmonary administration for the treatment of erectile dysfunction has been reported to be approximately 60 min. The elimination half life for a dose of apomorphine delivered by pulmonary administration for the treatment of Parkinson's Disease as disclosed herein was approximately 20-60 minutes.

Thus, a composition comprising apomorphine according to the present invention provides a Cₘₐₓ within 1 to 5 minutes of administration upon administration of the composition by pulmonary inhalation. The Cₘₐₓ is dose dependent. This rapid absorption of the apomorphine upon inhalation allows the administration of these compositions to provide a therapeutic effect in about 10 minutes or less.

The compositions according to the present invention also a terminal elimination half-life of between 30 and 70 minutes following pulmonary inhalation.

The significance of these pharmacokinetics for the compositions of the present invention is that they show that inhalation of the apomorphine compositions results in a consistent Tₘₐₓ of between 1 and 3 minutes with very little patient-to-patient variability. This is in contrast to the Tₘₐₓ observed following subcutaneous administration of apomorphine which varies from 10 to 60 minutes and exhibits great patient-to-patient variability.

### Preparing Dry Powder Inhaler Formulations

Where the compositions of the present invention include an additive material, the manner in which this is incorporated will have a significant impact on the effect that the additive material has on the powder performance, including the FPF and FPD.

In one embodiment, the compositions according to the present invention are prepared by simply blending particles of apomorphine of a selected appropriate size with particles of additive material and/or carrier particles. The powder components may be blended by a gentle mixing process, for example in a tumble mixer such as a Turbula (trade mark). In such a gentle mixing process, there is generally substantially no reduction in the size of the particles being mixed. In addition, the powder particles do not tend to become fused to one another, but they rather agglomerate as a result of cohesive forces such as Van der Waals forces. These loose or unstable agglomerates readily break up upon actuation of the inhaler device used to dispense the composition.

### Compressive Milling Processes

In an alternative process for preparing the compositions according to the present invention, the powder components undergo a compressive milling process, such as processes termed mechanofusion. (also known as ‘Mechanical Chemical Bonding’) and cyclomixing.

As the name suggests, mechanofusion is a dry coating process designed to mechanically fuse a first material onto a second material. It should be noted that the use of the terms “mechanofusion” and “mechanofused” are supposed to be interpreted as a reference to a particular type of milling process, but not a milling process performed in a particular apparatus. The compressive milling processes work according to a different principle to other milling techniques, relying on a particular interaction between an inner element and a vessel wall, and they are based on providing energy by a controlled and substantial compressive force. The process works particularly well where one of the materials is generally smaller and/or softer than the other.

The fine active particles and additive particles are fed into the vessel of a mechanofusion apparatus (such as a Mechano-Fusion system (Hosokawa Micron Ltd) or the Nobilta or Nanocular apparatus, where they are subject to a centrifugal force and are pressed against the vessel inner wall. The powder is compressed between the fixed clearance of the drum wall and a curved inner element with high relative speed between drum and element. The inner wall and the curved element together form a gap or nip in which the particles are pressed together. As a result, the particles experience very high shear forces and very strong compressive stresses as they are trapped between the inner drum wall and the inner element (which has a greater curvature than the inner drum wall). The particles are pressed against each other with enough energy to locally heat and soften, break, distort, flatten and wrap the additive particles around the core particle to form a coating. The energy is generally sufficient to break up agglomerates and some degree of size reduction of both components may occur.

These mechanofusion and cyclomixing processes apply a high enough degree of force to separate the individual particles of active material and to break up tightly bound agglomerates of the active particles such that effective mixing and effective application of the additive material to the surfaces of those particles is achieved. An especially desirable aspect of the processes is that the additive material becomes deformed in the milling and may be smeared over or fused to the surfaces of the active particles.

However, in practice, these compression milling processes produce little or no size reduction of the drug particles, especially where they are already in a micronised form (i.e. <10 μm). The only physical change which may be observed is a plastic deformation of the particles to a rounder shape.

### Other Milling Procedures

The process of milling may also be used to formulate the dry powder compositions according to the present invention. The manufacture of fine particles by milling can be achieved using conventional techniques. In the conventional use of the word, “milling” means the use of any mechanical process which applies sufficient force to the particles of active material that it is capable of breaking coarse particles (for example, particles with a MMAD greater than 100 μm) down to fine particles (for example, having a MMAD not more than 50 μm). In the present invention, the term “milling” also refers to deagglomeration of particles in a formulation, with or without particle size reduction. The particles being milled may be large or fine prior to the milling step. A wide range of milling devices and conditions are suitable for use in the production of the compositions of the inventions. The selection of appropriate milling conditions, for example, intensity of milling and duration, to provide the required degree of force will be within the ability of the skilled person.

Impact milling processes may be used to prepare compositions comprising apomorphine according to the present invention, with or without additive material. Such processes include ball milling and the use of a homogenizer.

Ball milling is a suitable milling method for use in the prior art co-milling processes. Centrifugal and planetary ball milling are especially preferred methods.

Alternatively, a high pressure homogeniser may be used in which a fluid containing the particles is forced through a valve at high pressure producing conditions of high shear and turbulence. Shear forces on the particles, impacts between the particles and machine surfaces or other particles, and cavitation due to acceleration of the fluid may all contribute to the fracture of the particles. Suitable homogenisers include EmulsiFlex high pressure homogenisers which are capable of pressures up to 4000 bar, Niro Soavi high pressure homogenisers (capable of pressures up to 2000 bar), and Microfluidics Microfluidisers (maximum pressure 2750 bar). The milling process can be used to provide the microparticles with mass median aerodynamic diameters as specified above. Homogenisers may be more suitable than ball mills for use in large scale preparations of the composite active particles. The milling step may, alternatively, involve a high energy media mill or an agitator bead mill, for example, the Netzsch high energy media mill, or the DYNO-mill (Willy A. Bachofen AG, Switzerland).

If a significant reduction in particle size is also required, co-jet milling is preferred, as disclosed in the earlier patent application published as WO 2005/025536. The co-jet milling process can result in composite active particles with low micron or sub-micron diameter, and these particles exhibit particularly good FPF and FPD, even when dispensed using a passive DPI.

The milling processes apply a high enough degree of force to break up tightly bound agglomerates of fine or ultra-fine particles, such that effective mixing and effective application of the additive material to the surfaces of those particles is achieved.

These impact processes create high-energy impacts between media and particles or between particles. In practice, while these processes are good at making very small particles, it has been found that neither the ball mill nor the homogenizer was particularly effective in producing dispersion improvements in resultant drug powders in the way observed for the compressive process. It is believed that the second impact processes are not as effective in producing a coating of additive material on each particle.

Conventional methods comprising co-milling active material with additive materials (as described in WO 2002/43701) result in composite active particles which are fine particles of active material with an amount of the additive material on their surfaces. The additive material is preferably in the form of a coating on the surfaces of the particles of active material. The coating may be a discontinuous coating. The additive material may be in the form of particles adhering to the surfaces of the particles of active material. Co-milling or co-micronising particles of active agent and particles of additive (FCA) or excipient will result in the additive or excipient becoming deformed and being smeared over or fused to the surfaces of fine active particles, producing composite particles made up of both materials. These resultant composite active particles comprising an additive have been found to be less cohesive after the milling treatment.

At least some of the composite active particles may be in the form of agglomerates. However, when the composite active particles are included in a pharmaceutical composition, the additive material promotes the dispersal of the composite active particles on administration of that composition to a patient, via actuation of an inhaler.

Milling may also be carried out in the presence of a material which can delay or control the release of the active agent.

The co-milling or co-micronising of active and additive particles may involve compressive type processes, such as mechanofusion, cyclomixing and related methods such as those involving the use of a Hybridiser or the Nobilta. The principles behind these processes are distinct from those of alternative milling techniques in that they involve a particular interaction between an inner element and a vessel wall, and in that they are based on providing energy by a controlled and substantial compressive force, preferably compression within a gap of predetermined width.

In one embodiment, if required, the microparticles produced by the milling step can then be formulated with an additional excipient. This may be achieved by a spray drying process, e.g. co-spray drying with excipients. In this embodiment, the particles are suspended in a solvent and co-spray dried with a solution or suspension of the additional excipient. Preferred additional excipients include trehalose, melezitose and other polysaccharides. Additional pharmaceutical effective excipients may also be used.

In another embodiment, the powder compositions are produced using a multi-step process. Firstly, the materials are milled or blended. Next, the particles may be sieved, prior to undergoing mechanofusion. A further optional step involves the addition of carrier particles. The mechanofusion step is thought to “polish” the composite active particles, further rubbing the additive material into the active particles. This allows one to enjoy the beneficial properties afforded to particles by mechanofusion, in combination with the very small particles sizes made possible by the jet milling.

The reduction in the cohesion and adhesion between the active particles can lead to equivalent performance with reduced agglomerate size, or even with individual particles.

### High Shear Blending

Scaling up of pharmaceutical product manufacture often requires the use one piece of equipment to perform more than one function. An example of this is the use of a mixer-granulator which can both mix and granulate a product thereby removing the need to transfer the product between pieces of equipment. In so doing, the opportunity for powder segregation is minimised. High shear blending often uses a high-shear rotor/stator mixer (HSM), which has become used in mixing applications. Homogenizers or “high shear material processors” develop a high pressure on the material whereby the mixture is subsequently transported through a very fine orifice or comes into contact with acute angles. The flow through the chambers can be reverse flow or parallel flow depending on the material being processed. The number of chambers can be increased to achieve better performance. The orifice size or impact angle may also be changed for optimizing the particle size generated. Particle size reduction occurs due to the high shear generated by the high shear material processors while it passes through the orifice and the chambers. The ability to apply intense shear and shorten mixing cycles gives these mixers broad appeal for applications that require agglomerated powders to be evenly blended. Furthermore conventional HSMs may also be widely used for high intensity mixing, dispersion, disintegration, emulsification and homogenization.

It is well known to those skilled in the production of powder formulations that small particles, even with high-power, high-shear, mixers a relatively long period of “aging” is required to obtain complete dispersion, and this period is not shortened appreciably by increases in mixing power, or by increasing the speed of rotation of the stirrer so as to increase the shear velocity. High shear mixers can also be used if the auto-adhesive properties of the drug particles are so that high shear forces are required together with use of a force-controlling agent for forming a surface-energy-reducing particulate coating or film.

### Spray Drying and Ultrasonic Nebulisers

Spray drying may be used to produce particles of inhalable size comprising the apomorphine. The spray drying process may be adapted to produce spray-dried particles that include the active agent and an additive material which controls the agglomeration of particles and powder performance. The spray drying process may also be adapted to produce spray-dried particles that include the active agent dispersed or suspended within a material that provides the controlled release properties. Furthermore the dispersal or suspension of the active material within an excipient material may impart further stability to the active compounds. In a preferred embodiment the apomorphine may reside primarily in the amorphous state. A formulation containing amorphous apomorphine will possess preferable dissolution characteristics. This would be possible in that particles are suspended in a sugar glass which could be either a solid solution or a solid dispersion. Preferred additional excipients include trehalose, melezitose and other polysaccharides.

Spray drying is a well-known and widely used technique for producing particles of active material of inhalable size. Conventional spray drying techniques may be improved so as to produce active particles with enhanced chemical and physical properties so that they perform better when dispensed from a DPI than particles formed using conventional spray drying techniques. Such improvements are described in detail in the earlier patent application published as WO 2005/025535.

In particular, it is disclosed that co-spray drying an active agent with an FCA under specific conditions can result in particles with excellent properties which perform extremely well when administered by a DPI for inhalation into the lung.

It has been found that manipulating or adjusting the spray drying process can result in the FCA being largely present on the surface of the particles. That is, the FCA is concentrated at the surface of the particles, rather than being homogeneously distributed throughout the particles. This clearly means that the FCA will be able to reduce the tendency of the particles to agglomerate. This will assist the formation of unstable agglomerates that are easily and consistently broken up upon actuation of a DPI.

It has been found that it may be advantageous to control the formation of the droplets in the spray drying process, so that droplets of a given size and of a narrow size distribution are formed. Furthermore, controlling the formation of the droplets can allow control of the air flow around the droplets which, in turn, can be used to control the drying of the droplets and, in particular, the rate of drying. Controlling the formation of the droplets may be achieved by using alternatives to the conventional 2-fluid nozzles, especially avoiding the use of high velocity air flows.

In particular, it is preferred to use a spray drier comprising a means for producing droplets moving at a controlled velocity and of a predetermined droplet size. The velocity of the droplets is preferably controlled relative to the body of gas into which they are sprayed. This can be achieved by controlling the droplets' initial velocity and/or the velocity of the body of gas into which they are sprayed, for example by using an ultrasonic nebuliser (USN) to produce the droplets. Alternative nozzles such as electrospray nozzles or vibrating orifice nozzles may be used.

In one embodiment, a USN is used to form the droplets in the spray mist. USNs use an ultrasonic transducer which is submerged in a liquid. The ultrasonic transducer (a piezoelectric crystal) vibrates at ultrasonic frequencies to produce the short wavelengths required for liquid atomisation. In one common form of USN, the base of the crystal is held such that the vibrations are transmitted from its surface to the nebuliser liquid, either directly or via a coupling liquid, which is usually water. When the ultrasonic vibrations are sufficiently intense, a fountain of liquid is formed at the surface of the liquid in the nebuliser chamber. Droplets are emitted from the apex and a “fog” emitted.

Whilst USNs are known, these are conventionally used in inhaler devices, for the direct inhalation of solutions containing drug, and they have not previously been widely used in a spray drying apparatus. It has been discovered that the use of such a nebuliser in spray drying has a number of important advantages and these have not previously been recognised. The preferred USNs control the velocity of the particles and therefore the rate at which the particles are dried, which in turn affects the shape and density of the resultant particles. The use of USNs also provides an opportunity to perform spray drying on a larger scale than is possible using conventional spray drying apparatus with conventional types of nozzles used to create the droplets, such as 2-fluid nozzles.

The attractive characteristics of USNs for producing fine particle dry powders include: low spray velocity; the small amount of carrier gas required to operate the nebulisers; the comparatively small droplet size and narrow droplet size distribution produced; the simple nature of the USNs (the absence of moving parts which can wear, contamination, etc.); the ability to accurately control the gas flow around the droplets, thereby controlling the rate of drying; and the high output rate which makes the production of dry powders using USNs commercially viable in a way that is difficult and expensive when using a conventional two-fluid nozzle arrangement.

USNs do not separate the liquid into droplets by increasing the velocity of the liquid. Rather, the necessary energy is provided by the vibration caused by the ultrasonic nebuliser.

Further embodiments, may employ the use of ultrasonic nebuliser (USN), rotary atomisers or electrohydrodynamic (EHD) atomizers to generate the particles.

### Delivery Devices

The inhalable compositions in accordance with the present invention are preferably administered via a dry powder inhaler (DPI), but can also be administered via a pressurized metered dose inhaler (pMDI), or even via a nebulised system.

In a dry powder inhaler, the dose to be administered is stored in the form of a non-pressurized dry powder and, on actuation of the inhaler, the particles of the powder are expelled from the device in the form of a cloud of finely dispersed particles that may be inhaled by the patient.

Dry powder inhalers can be “passive” devices in which the patient's breath is the only source of gas which provides a motive force in the device. Examples of “passive” dry powder inhaler devices include the Rotahaler and Diskhaler (GlaxoSmithKline), the Monohaler (MIAT), the GyroHaler (trademark) (Vectura) the Turbohaler (Astra-Draco) and Novolizer (trade mark) (Viatris GmbH). Alternatively, “active” devices may be used, in which a source of compressed gas or alternative energy source is used. Examples of suitable active devices include Aspirair (trade mark) (Vectura) and the active inhaler device produced by Nektar Therapeutics (as covered by U.S. Pat. No. 6,257,233).

It is generally considered that different compositions perform differently when dispensed using passive and active type inhalers. Passive devices create less turbulence within the device and the powder particles are moving more slowly when they leave the device. This leads to some of the metered dose remaining in the device and, depending on the nature of the composition, less deagglomeration upon actuation. However, when the slow moving cloud is inhaled, less deposition in the throat is often observed. In contrast, active devices create more turbulence when they are activated. This results in more of the metered dose being extracted from the blister or capsule and better deagglomeration as the powder is subjected to greater shear forces. However, the particles leave the device moving faster than with passive devices and this can lead to an increase in throat deposition.

It has been surprisingly found that the compositions of the present invention with their high proportion of apomorphine perform well when dispensed using both active and passive devices. Whilst there tends to be some loss along the lines predicted above with the different types of inhaler devices, this loss is minimal and still allows a substantial proportion of the metered dose of apomorphine to be deposited in the lung. Once it reaches the lung, the apomorphine is rapidly absorbed and exhibits excellent bioavailability.

Particularly preferred “active” dry powder inhalers are referred to herein as Aspirair® inhalers and are described in more detail in WO 2001/00262, WO 2002/07805, WO 2002/89880 and WO 2002/89881, the contents of which are hereby incorporated by reference. It should be appreciated, however, that the compositions of the present invention can be administered with either passive or active inhaler devices.

In an alternative embodiment, the composition is a solution or suspension, which is dispensed using a pressurised metered dose inhaler (pMDI). The composition according to this embodiment can comprise the dry powder composition discussed above, mixed with or dissolved in a liquid propellant such as HFA 134a or HFA 227.

In a yet further embodiment, the composition is a solution or suspension and is administered using a pressurised metered dose inhaler (pMDI), a nebuliser or a soft mist inhaler. Examples of suitable devices include pMDIs such as Modulite® (Chiesi), SkyeFine™ and SkyeDry™ (SkyePharma). Nebulisers such as Porta-Neb®, Inquaneb™ (Pari) and Aquilon™, and soft mist inhalers such as eFlow™ (Pari), Aerodose™ (Aerogen), Respimat® Inhaler (Boehringer Ingelheim GmbH), AERx® Inhaler (Aradigm) and Mystic™ (Ventaira Pharmaceuticals, Inc.).

Where the composition is to be dispensed using a pMDI, the composition comprising apomorphine preferably further comprises a propellant. In embodiments of the present invention, the propellant is CFC-12 or an ozone-friendly, non-CFC propellant, such as 1,1,1,2-tetrafluoroethane (HFC 134a), 1,1,1,2,3,3,3-heptafluoropropane (HFC-227), HCFC-22 (difluororchloromethane), HFA-152 (difluoroethane and isobutene) or combinations thereof. Such formulations may require the inclusion of a polar surfactant such as polyethylene glycol, diethylene glycol monoethyl ether, polyoxyethylene sorbitan monolaurate, polyoxyethylene sorbitan monooleate, propoxylated polyethylene glycol, and polyoxyethylene lauryl ether for suspending, solubilizing, wetting and emulsifying the active agent and/or other components, and for lubricating the valve components of the MDI.

### SUMMARY

In conclusion the advantages of pulmonary delivery may be summarised as follows.

The increased delivery efficiency and bioavailability achieved by pulmonary delivery present the opportunity to achieve required efficacy at an apomorphine dose level approximately three-times lower than that studied with intranasal delivery and ultimately a superior risk:benefit profile.

Pulmonary delivery via oral inhalation, not being subject to some of the complexities surrounding nasal administration, results in more rapid and consistent systemic exposure which translates to an accelerated and surprisingly predictable therapeutic response. These parameters are key unmet clinical needs when considering the treatment of many disorders of the central nervous system, and Parkinson's Disease in particular.

Pulmonary delivery of apomorphine constitutes a more patient friendly administration route, which is associated with a superior local tolerability profile, with no evidence of the administration site adverse events reported with intranasal delivery.

### EXAMPLES

### Example 1

### Spray Dried Apomorphine

Feasibility batch: Apomorphine hydrochloride (5.04 g, Batch No. GRN 0436) was dissolved in 250 ml purified water resulting in a 2% w/v total solids feedstock. The feedstock was spray dried using a bespoke Mini Spray Dryer with an inlet temperature of 155° C. and an atomisation pressure of 3 bar. The geometric particle size of the resultant spray dried powder (Batch No. RDD/07/095) was determined using a Sympatec Particle Size Analyser, the mean of three analyses was as follows:

### X10 (μm): 1.05

### X50 (μm): 1.91

### X90 (μm): 3.15

99 (μm): 4.12

Scale up batch: Apomorphine hydrochloride (14.9 g, Batch No. GRN 0436) was dissolved in 750 ml purified water resulting in a 2% w/v total solids feedstock. The feedstock was spray dried using a bespoke Mini Spray Dryer with an inlet temperature of 155° C. and an atomisation pressure of 3 bar. The geometric particle size of the resultant spray dried powder (Batch No. RDD/07/096) was determined using a Sympatec Particle Size Analyser, the mean of three analyses was as follows:

### X10 (μm): 1.10

### X50 (μm): 2.10

90 (μm): 3.49
99 (μm): 4.43

### Example 2

### pMDIs

Preparation of pMDIs: the Powders Comprising Pure Micronised Apomorphine hydrochloride were measured into pMDI cans. Metering valves were clamped onto the cans, and these were back filled with HFA 134a propellant. Each can was shaken vigorously to generate a dispersion.

In Vitro measurement of pMDIs: An Andersen cascade impactor was used to characterise the aerosol plumes generated from each of the pMDIs. Air-flow of 28.3 litres per minute was drawn through the impactor, and 10 repeated shots were fired. Each pMDI was shaken and weighed in between each actuation. The drug deposited on each stage of the impactor, as well as drug on the device, throat and rubber mouthpiece adaptor was collected into a solvent, and quantified by HPLC.

The low solubility of apomorphine hydrochloride within ethanol-based HFA 134a pMDI formulations makes solution pMDI technology unavailable for apomorphine at high drug loading (600 μg/dose). Previously a low dose (<25 μg/50 μl) HFA134a/HFA 227 solution formulation has been produced but only at high ethanol contents (50% w/w). An apomorphine analogue may be used to formulate highly efficient solution formulations at the desirable dose range of 100 to 500 μg/50 μl.

Nine formulations (see Table 1 and Table 2) were manufactured.

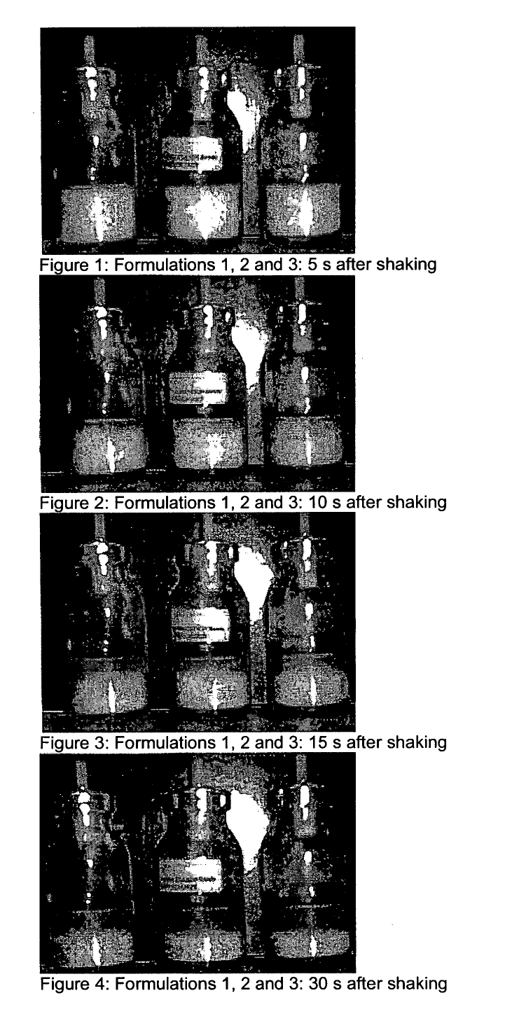

Visual assessment of 600 μg/50 μl formulations (see FIGS. 1-4) found that the apomorphine rapidly sedimented in pure HFA134a and that small additions of absolute ethanol (5% w/w) and oleic acid (0.04% w/w) did not noticeably slow the sedimentation rate.

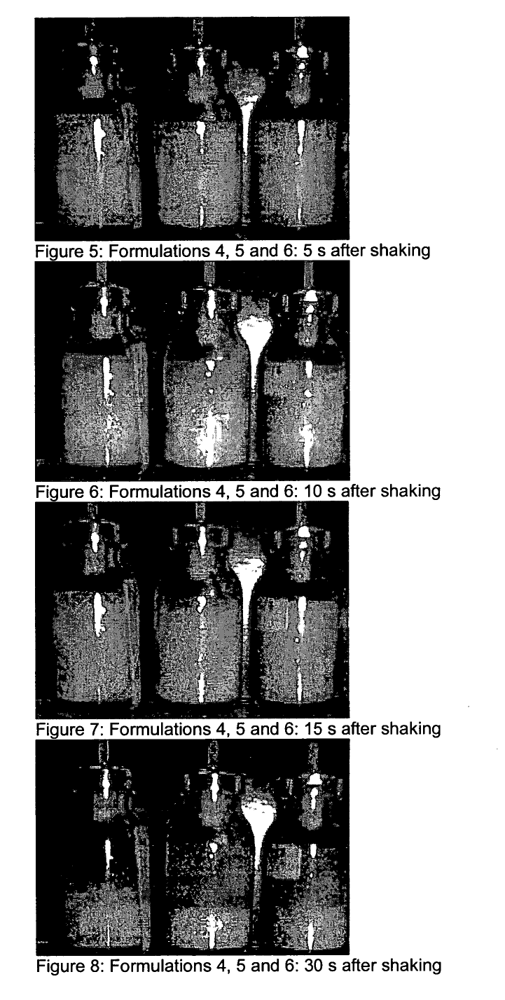

Reduction of the drug concentration (300 μg/50 μl) was investigated in Formulations 4-6 (see FIGS. 5-8). Apomorphine was again observed to rapidly sediment in pure HFA134a. Small additions of absolute ethanol (2.5% w/w) and oleic acid (0.02% w/w) did not noticeably slow the sedimentation rate.

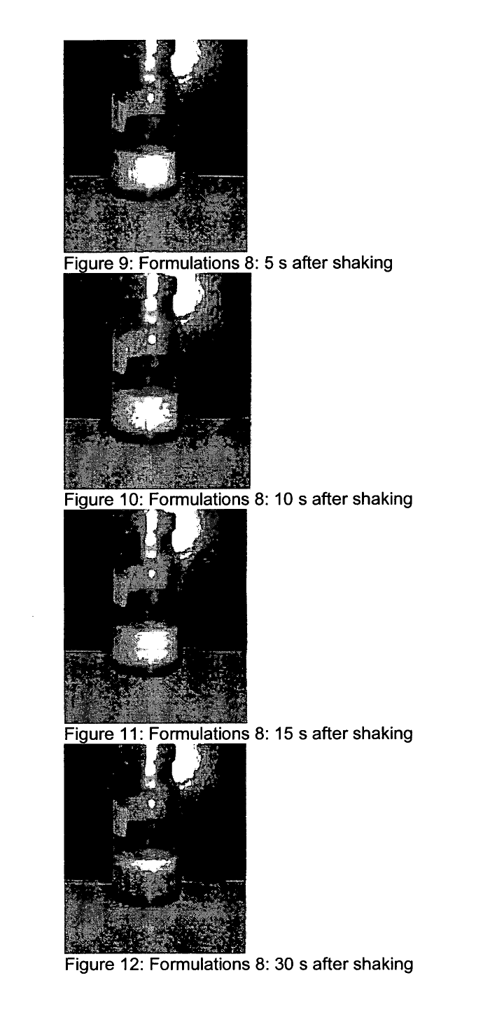

When a HFA 227 apomorphine (264 μg/50 μl) suspension was manufactured (see Table 2) apomorphine was observed to cream (float) (see FIGS. 9-12) indicating that the density of the apomorphine particles is somewhere between that of HFA134a (1.226 g/ml) and HFA 227 (1.415 g/ml). The addition of HFA134a to the HFA 227 suspension allowed the density of the apomorphine to be matched at a composition of approximately 60(% w/w) FIFA 227 and 40(% w,/w) HFA134a indicating that the density of apomorphine is about 1.33 g/ml.

It may be possible to develop a highly volatile suspension formulation using a combination of HFA 227 & HFA 134a at a 60:40(% w/w) ratio. The high volatility of the formulation could lead to highly efficient atomisation and good <5 μm delivery. It is expected that the formulation will be compatible with Bespak BK630 series 0.22-0.30 mm actuators, although small amounts of ethanol (2% w/w) may be required to suppress rapid propellant flashing near the actuator orifice such that blockage (if a problem) can be addressed. The use of 3M coated (Dupont 3200 200) cans and Valois DF31 50 μl valves should function well with this type of formulation and facilitate consistent through can life delivery performance. The lack of excipients may lead to good formulation stability.

TABLE 1
Apomorphine, details of formulations manufactured (all formulations
ultrasonicated for two minutes before visual assessment).

| | | | Oleic | HFA | Estimated | |
|---|---|---|---|---|---|---|
| | Drug | Ethanol | Acid | 134a | Vol | Drug |
| Formulation | (mg) | (mg) | (mg) | (mg) | (ml) | (μg/50 μl) |
| 1 | 72.9 | 0 | 0 | 7704.3 | 6.3 | 574 |
| 2 | 73.7 | 384.2 | 0 | 6810.1 | 6.1 | 604 |
| 3 | 70.7 | 364.3 | 2.7 | 6756.4 | 6.0 | 586 |
| 4 | 72.9 | 0 | 0 | 15052.5 | 12.3 | 295 |
| 5 | 73.7 | 384.2 | 0 | 13957.6 | 11.9 | 309 |
| 6 | 70.7 | 364.3 | 2.7 | 14023.5 | 12.0 | 296 |

TABLE 2
Apomorphine HFA 227 and HFA134a combination formulations
(all formulations were ultrasonicated for two
minutes before visual assessment).

| | | | HFA | HFA | Estimated | |
|---|---|---|---|---|---|---|
| | Drug | Ethanol | 134a | 227 | Vol | Drug |
| Formulation | (mg) | (mg) | (mg) | (mg) | (ml) | (μg/50 μl) |
| 8 | 26.7 | 0 | 0 | 7129.7 | 5.1 | 264 |
| 9 | 26.7 | 0 | 3023.2 | 7129.7 | 7.5 | 177 |
| 10 | 26.7 | 0 | 4229 | 7129.7 | 8.5 | 157 |

1.3415 g/ml

TABLE 3
Results

| | | | | Shot | | | |
|---|---|---|---|---|---|---|---|
| Formu- | MD | FPD | MMAD | Weight | DD | FPF | |
| lation | (μg) | (μg) | (μg) | (mg) | (μg) | (%) | GSD |
| 2 | 517.42 | 314.14 | 3.47 | 63.9 | 470.96 | 66.70 | 1.48 |

### Example 3

### Active/Passive DPIs Mechanofused Apomorphine with Magnesium Stearate Formulations that are Subsequently Combined

Combined formulations i.e. comprising different particles:

(a) Apomorphine hydrochloride with magnesium stearate covering:

Micronised apomorphine hydrochloride and magnesium stearate were combined in a weight ratio of 75:25. This blend (˜20 g) was then milled by a mechanofusion process as follows. The powder was pre-mixed for 5 minutes at −900 rpm. The machine speed was then increased to ˜4,800 rpm for 30 minutes. During the milling treatment the mechanofusion apparatus is run with a 1 mm clearance between element and vessel wall, and with cooling water applied via the cooling jacket. The composite active particles were then recovered from the drum vessel.

(b) Apomorphine hydrochloride with less magnesium stearate covering:

The experiment was repeated using the same procedure but the active particle and homogenised magnesium stearate were combined in the ratio 95:5, and milled for 60 minutes at 4,800 rpm.

(c) Combine formulations (a) and (b) together to obtain rapid onset from Formulation (b) and delayed dissolution from Formulation (a):

Samples of the apomorphine hydrochloride formulations (a) and (b) were mixed in a Turbula Mixer for 10 minutes at a speed of 32 rpm.

(d) Apomorphine formulation with microparticulate additive on the surface of the apomorphine particles to reduce interparticulate cohesion and upon actuation from a dry powder inhaler will result in an extended inhaled bolus.

### Example 4

### Lactose Formulation—30% Micronised Apomorphine HCl with 70% Lactose (45-63 μm)

The lactose was sieved to give samples with particles with a range of diameter from 45-63 μm. The first sieve screen size used was 63 μm. Successive samples of approximately 500 ml were sieved mechanically for 5 minutes. The second sieve screen size used was 45 μm. Successive samples of approximately 250 ml were sieved mechanically for 10 minutes. To prevent blinding of the sieve by the lactose particles the 45 μm screen was vacuumed after two samples. Samples were taken from those particles which had passed through the 63 μm sieve but remained on top of the 45 μm sieve. Those particles could be considered to have a diameter between 45-63 μm.

Samples of the lactose particles obtained in the above step were treated by mixing the lactose particles with active particles of apomorphine hydrochloride (particle size d0.5: 2.2 μm). A 210 g sample of the lactose particles and 90 g sample of the active apomorphine hydrochloride particles were placed into the 2 L volume Diosna bowl by transferring approximately 50% of the lactose and adding all of the apomorphine hydrochloride, the remaining 50% was placed on top sandwiching the active particles.

The lactose and apomorphine hydrochloride particles were pre-mixed using the Diosna mixer for 72 seconds at 214 rpm with the chopper set at 30 rpm. The particles were then mixed for 7 minutes at 857 rpm with the chopper set at 30 rpm, this process was stopped at 1 minute intervals and the sides of the bowl scraped down. The mixture was passed manually through a 315 μm sieve screen. The mixture was returned to the Diosna and mixed for 72 seconds at 214 rpm with the chopper set at 30 rpm.

TABLE 4
Performance results

Active device
Aspirair ®

| | | |
|---|---|---|
| Nominal Dose (μg) | 3200 | 4500 |
| Delivered Dose (μg) | 2465 | 3486 |
| Fine Particle Dose ≦5 μm (μg) | 1509 | 1890 |
| Fine Particle Fraction ≦5 μm (%) | 61.2 | 54.3 |
| Fine Particle Dose ≦3 μm (μg) | 1097 | 1297 |
| Fine Particle Fraction ≦3 μm (%) | 44.5 | 37.3 |
| MMAD (μm) | 2.4 | 2.5 |
| GSD | 1.6 | 1.7 |

Pharmacokinetic results: Apomorphine was rapidly absorbed with peak apomorphine plasma concentration observed 1-3 minutes post-inhalation. Dose proportionality was observed for AUC₍₀₋ₜ₎, AUC₍₀₋ₘ₎ and Cₘₐₓ.

TABLE 5
Results

| | | Treatment Group | | |
|---|---|---|---|---|
| | | Apomorphine | Apomorphine | Apomorphine |
| Parameter | | 400 μg (N = 6) | 1000 μg (N = 6) | 1600 μg (N = 6) |
| AUC₍₀₋ₜ₎ | Mean (SD) | 47.50 (10.04) | 168.81 (90.20) | 270.75 (37.97) |
| (ng · min/mL) | | | | |
| AUC_{(0-∞)} | Mean (SD) | 56.70 (12.94) | 216.93 (99.30) | 301.57 (36.64) |
| (ng · min/mL) | | | | |
| Cₘₐₓ (ng/mL) | Mean (SD) | 3.03 (0.71) | 10.0 (8.39) | 11.92 (1.17) |
| k | Mean (SD) | 0.01 (0.00) | 0.01 (0.00) | 0.02 (0.00) |
| t_{1/2} | Mean (SD) | 37.27 (8.90) | 37.10 (5.54) | 25.70 (2.47) |
| tₘₐₓ | Mean (SD) | 1.0 (0.0) | 2.6 (2.6) | 2.2 (1.1) |

Note:
The 400 μg dose was a 20% apomorphine in a 80% lactose blend. The 1000 and 1600 μg doses were 30% apomorphine in a 70% lactose blend.

These results suggest a dose-proportional 3- to 5.7-fold increase in the apomorphine plasma concentration.

Safety results: Few patients experienced treatment emergent adverse events (TEAEs) and there were no notable differences in incidence or types of TEAEs among the treatment groups. Nominal Doses of 400 μg, 1000 μg and 1600 μg were well tolerated. Most common TEAEs at <24 h post dose were nervous system disorders.

### Example 5

### 90% Micronised Apomorphine Hydrochloride with 10% Magnesium Stearate—Active/Passive DPIs

Samples of the active particles of apomorphine hydrochloride were treated by mixing with particles of magnesium stearate. 40 g of magnesium stearate particles were added to 360 g of apomorphine hydrochloride particles (particle size d_{0.5}: 2.2 μm) and mixed in a Turbula Mixer for 10 minutes at a speed of 32 rpm.

The mixture was sieved by passing through a 315 μm sieve screen. The mixture was then passed through a jet mill at a rate of 5 g/min using 8 bar venturi pressure and 5 bar grind pressure. The mixture was then passed manually through a 315 μm sieve screen.

TABLE 6
Performance data

| | Active device - | Passive device - |
|---|---|---|
| | Aspirair ® | Monohaler ® |
| Nominal Dose (μg) | 5400 | 5400 |
| Delivered Dose (μg) | 4416 | 4011 |
| Fine Particle Dose ≦5 μm | 3960 | 3418 |
| (μg) | | |
| Fine Particle Fraction ≦5 μm | 89.7 | 85.4 |
| (%) | | |
| Fine Particle Dose ≦3 μm | 3206 | 3027 |
| (μg) | | |
| Fine Particle Fraction ≦3 μm | 72.6 | 75.4 |
| (%) | | |
| MMAD (μm) | 2.1 | 2.0 |
| GSD | 1.6 | 1.5 |

### Example 6

### 50% Jet Milled Apomorphine Hydrochloride and Magnesium Stearate (Example 2) Mixed with 50% Lactose Mechanofused with Magnesium Stearate

(a) Extrafine lactose (with an approximate particle size d_{0.5} of 30 μm) was mechanofused with 5% magnesium stearate using the Hosokawa Nanocular.

(b) Samples (5 g) of carrier particles produced in step (a) containing lactose and 5% by weight particles of magnesium stearate were combined with 5 g samples of the mixture from Example 5 by high shear mixing for 10 minutes. Several 12 mg samples of the mixture were transferred to Aspirair® blisters for in-vitro assessment using the Aspirair® dry powder inhaler.

TABLE 7
Results

| | Active device | Passive device |
|---|---|---|
| | Aspirair ® | Monohaler ® |
| Nominal Dose (μg) | 5400 | 5400 |
| Delivered Dose (μg) | 4325 | 4616 |
| Fine Particle Dose ≦5 μm (μg) | 3058 | 4171 |
| Fine Particle Fraction ≦5 μm (%) | 70.7 | 90.4 |
| Fine Particle Dose ≦3 μm (μg) | 2428 | 3682 |
| Fine Particle Fraction ≦3 μm (%) | 56.1 | 79.8 |
| MMAD (μm) | 2.2 | 2.0 |
| GSD | 1.7 | 1.5 |

### Example 7

### Co-Jet Milled 98% Micronised Apomorphine Hydrochloride with 2% Leucine

Samples of the active particles of apomorphine hydrochloride were treated by mixing with particles of leucine. 3 g of leucine particles were added to 147 g of apomorphine hydrochloride particles (particle size d_{0.5}: 2.2 μm) and mixed in a Turbula Mixer for 10 minutes at a speed of 32 rpm.

The mixture was sieved by passing through a 315 μm sieve screen. The mixture was passed through a jet mill at a rate of 5 g/min using 8 bar venturi pressure and 5 bat grind pressure. The mixture was sieved manually through a 315 μm sieve screen.

Several 6 mg samples of the mixture were transferred to Aspirair® blisters for in-vitro assessment using the Aspirair® dry powder inhaler.

TABLE 8
Results

| | Aspirair ® |
|---|---|
| Nominal Dose (μg) | 5400 |
| Delivered Dose (μg) | 3583 |
| Fine Particle Dose ≦5 μm (μg) | 2380 |
| Fine Particle Fraction ≦5 μm (%) | 66.5 |
| Fine Particle Dose ≦3 μm (μg) | 1689 |
| Fine Particle Fraction ≦3 μm (%) | 47.2 |
| MMAD (μm) | 2.4 |
| GSD | 1.6 |

### Example 8

### Diosna Blend

Apomorphine particles were prepared using a Hosakowa AS100 Jet Mill resulting in a D_{0.5} of 1.9 μm. To manufacture the final formulation comprising Apomorphine 18.5% (w/w), magnesium stearate 1.5% (w/w) and Lactose (Respitose SV003) 80% (w/w), the three components were screened separately with a Quadro® Comil® using a 813 μm screen size at a speed of 1000 rpm until completion. A pre-blend was made of the lactose and magnesium stearate using the Diosna mixer at 1500 rpm for 1 minute.

Approximately 50% of the lactose and magnesium stearate pre-blend was removed from the Diosna bowl and a sample of the active apomorphine hydrochloride was placed on top of the remaining lactose and magnesium stearate pre-blend. The removed lactose and magnesium stearate pre-blend was then replaced on top of the apomorphine hydrochloride layer thereby “sandwiching” the active particles. This formulation was then processed at 600 rpm for 6 minutes.

The completed formulation was filled into blisters with an Omnidose filling machine and loaded into a passive device. The formulation was assessed using an Anderson Cascade Impactor at 57 L/minute with 5 actuations per assessment.

TABLE 9
Results

| | Dry Powder Inhaler |
|---|---|
| Nominal Dose (μg) | 7722 |
| Delivered Dose (μg) | 6808 |
| Fine Particle Dose ≦5 μm (μg) | 3808 |
| Fine Particle Fraction ≦5 μm (%) | 55.9 |
| Fine Particle Dose ≦3 μm (μg) | 2729 |
| Fine Particle Fraction ≦3 μm (%) | 40.1 |
| MMAD (μm) | 2.4 |
| GSD | 1.8 |

### Example 9

### PowderHale Formulation: Co-Jet Milling Followed by MCB

Samples of the active particles of apomorphine hydrochloride were treated by mixing with particles of magnesium stearate. 40 g of magnesium stearate particles are added to 360 g of apomorphine hydrochloride particles (particle size d_{0.5}: 2.2 μm) and mixed in a Turbula Mixer for 10 minutes at a speed of 32 rpm.

(a) The mixture was sieved by passing it through a 315 μm sieve screen. The mixture was then passed through a Jet Mill (Hosakowa AS50S) at a rate of 5 g/min using 8 bar venturi pressure and 5 bar grind pressure. The mixture was then passed manually through a 315 μm sieve screen.

(b) Samples (80 mL) of co-jet milled formulation (a) were milled by a mechanofusion process as follows. The machine was initially run at 5% of the maximum speed for 5 minutes. The machine speed was then increased to 20% of the maximum speed for 5 minutes. Finally the machine was run at 80% of the maximum speed for 10 minutes. During the milling treatment the mechanofusion apparatus was run with a 3 mm clearance between element and vessel wall with the cooling water applied via the cooling jacket. The resultant active particles were recovered from the drum vessel.

The completed formulation was filled into blisters by hand and loaded into an Aspirair® device. The formulation was assessed using an Anderson Cascade Impactor at 60 L/minute with 5 actuations per assessment.

TABLE 10
Results

| | Aspirair ® |
|---|---|
| Nominal Dose (μg) | 5400 |
| Delivered Dose (μg) | 4334 |
| Fine Particle Dose ≦5 μm (μg) | 3859 |
| Fine Particle Fraction ≦5 μm (%) | 89.1 |
| Fine Particle Dose ≦3 μm (μg) | 3252 |
| Fine Particle Fraction ≦3 μm (%) | 75.2 |
| MMAD (μm) | 1.9 |

### Example 10

### Co-Jet Milling Followed and High Shear Blending with Lactose

(a) Samples of the active particles of apomorphine hydrochloride were treated by mixing with particles of magnesium stearate. 40 g of magnesium stearate particles were added to 360 g of apomorphine hydrochloride particles (particle size d_{0.5}: 2.2 μm) and mixed in a Turbula Mixer for 10 minutes at a speed of 32 rpm.

The mixture was sieved by passing through a 315 μm sieve screen. The mixture was then passed through a Jet Mill (Hosakowa AS50S) at a rate of 5 g/min using 8 bar venturi pressure and 5 bar grind pressure. The mixture was then passed manually through a 315 μm sieve screen.

(b) A sample of 33 g of co-jet milled formulation (a) and 117 g of lactose particles were placed into the 1 L volume Diosna bowl by transferring approximately 50% of the lactose and adding all of (a), the remaining 50% was placed on top sandwiching the co-jet milled particles. The lactose and (a) were pre-mixed using the Diosna mixer for 1 minute at 214 rpm with the chopper set at 30 rpm. The particles were then mixed for 6 minutes at 1000 rpm with the chopper set at 30 rpm, this process was stopped at 1 minute intervals and the sides of the bowl scraped down. The mixture was passed manually through a 160 μm sieve screen. The mixture was returned to the Diosna and mixed for 1 minute at 250 rpm with the chopper set at 30 rpm.

The completed formulation was filled into blisters by hand and loaded into a passive device. The formulation was assessed using an Anderson Cascade Impactor at 57 L/minute with 5 actuations per assessment.

TABLE 11
Results

| | Dry Powder Inhaler |
|---|---|
| Nominal Dose (μg) | 7200 |
| Delivered Dose (μg) | 6382 |
| Fine Particle Dose ≦5 μm (μg) | 3544 |
| Fine Particle Fraction ≦5 μm (%) | 55.6 |
| Fine Particle Dose ≦3 μm (μg) | 3201 |
| Fine Particle Fraction ≦3 μm (%) | 50.3 |
| MMAD (μm) | 1.5 |

### Example 11

### Co-Jet Milling Followed by MCB is then High Shear Blended with Lactose

(a) Samples of the active particles of apomorphine hydrochloride were treated by mixing with particles of magnesium stearate. 40 g of magnesium stearate particles were added to 360 g of apomorphine hydrochloride particles (particle size d_{0.5}: 2.2 μm) and mixed in a Turbula Mixer for 10 minutes at a speed of 32 rpm.

The mixture was sieved by passing through a 315 μm sieve screen. The mixture was then passed through a Jet Mill (Hosakowa AS50S) at a rate of 5 g/min using 8 bar venturi pressure and 5 bar grind pressure. The mixture was then passed manually through a 315 μm sieve screen.

(b) Samples (80 ml) of the co-jet Milled formulation (a) was milled according to the following mechanofusion process. The machine was initially run at 5% of the maximum speed for 5 minutes. The machine speed was then increased to 20% of the maximum speed for 5 minutes. Finally the machine was run at 80% of the maximum speed for 10 minutes. During the milling treatment the mechanofusion apparatus was run with a 3 mm clearance between element and vessel wall and with the cooling water applied via the cooling jacket. The resultant active particles were then recovered from the drum vessel.

(c) A sample of 33 g of co-jet milled formulation (a) and 117 g of lactose particles were placed into the 1 L volume Diosna bowl by transferring approximately 50% of the lactose and adding all of (a), the remaining 50% was placed on top thereby sandwiching the co-jet milled particles. The lactose and (a) were pre-mixed using the Diosna mixer for 1 minute at 214 rpm with the chopper set at 30 rpm. The particles were then mixed for 6 minutes at 1000 rpm with the chopper set at 30 rpm, this process was stopped at 1 minute intervals and the sides of the bowl scraped down. The mixture was passed manually through a 160 μm sieve screen. The mixture was returned to the Diosna and mixed for 1 minute at 250 rpm with the chopper set at 30 rpm.

The completed formulation was filled into blisters by hand and loaded into a passive device. The formulation was assessed using an Anderson Cascade Impactor at 57 L/minute with 5 actuations per assessment.

TABLE 12
Results:

| | Dry Powder Inhaler |
|---|---|
| Nominal Dose (μg) | 7200 |
| Delivered Dose (μg) | 5851 |
| Fine Particle Dose ≦5 μm (μg) | 4328 |
| Fine Particle Fraction ≦5 μm (%) | 74.0 |
| Fine Particle Dose ≦3 μm (μg) | 3916 |
| Fine Particle Fraction ≦3 μm (%) | 67.0 |
| MMAD (μm) | 1.6 |

### Example 12

### Lactose and Magnesium Stearate

(a) Samples of lactose particles and magnesium stearate particles were screened through a 813 μm screen using a Quadro® Comil® at 1000 rpm. Samples of lactose particles were treated by mixing the lactose particles with particles of magnesium stearate. A 480 g sample of lactose particles and 12 g sample of magnesium stearate particles were placed into the 2 L volume Diosna bowl by transferring approximately 50% of the lactose and adding all of the magnesium stearate, the remaining 50% of the lactose is placed on top sandwiching the magnesium stearate. The lactose and magnesium stearate particles were mixed using the Diosna mixer for 1 minute at 1500 rpm.

(b) A sample of active apomorphine hydrochloride particles was screened through a 813 μm screen using a Quadro® Comil® at 1000 rpm. Approximately 50% of (a) was removed from the Diosna bowl, and a sample of 108 g of active apomorphine hydrochloride particles placed into the Diosna bowl, the removed material (a) placed on top sandwiching the active particles. The contents of the Diosna bowl were mixed for 6 minutes at 500 rpm. The resultant mixture was then recovered from the Diosna bowl.

The completed formulation was filled into blisters by hand and loaded into a passive device. The formulation was assessed using an Anderson Cascade Impactor at 57 L/minute with 5 actuations per assessment.

TABLE 13
Results

| | Dry Powder Inhaler |
|---|---|
| Nominal Dose (μg) | 9000 |
| Delivered Dose (μg) | 7634 |
| Fine Particle Dose ≦5 μm (μg) | 4451 |
| Fine Particle Fraction ≦5 μm (%) | 58.3 |
| Fine Particle Dose ≦3 μm (μg) | 2985 |
| Fine Particle Fraction ≦3 μm (%) | 39.1 |
| MMAD (μm) | 2.7 |

1. A dry powder composition comprising apomorphine for administration by pulmonary inhalation, for treating conditions of the central nervous system, including Parkinson's Disease.

2. The composition according to claim 1, comprising a dose of apomorphine up to 15 mg and at least 1 mg.

3. The composition according to claim 2, wherein the dose is a nominal dose.

4. The composition according to claim 1, wherein the composition provides a fine particle fraction (FPF) dose of about 2 to about 6 mg upon administration.

5. The composition according to claim 1, wherein doses of the apomorphine composition are to be administered to the patient as needed.

6. The composition according to claim 1, wherein doses may be administered sequentially, with the effect of each dosing being assessed by the patient before the next dose is administered to allow self-titration and optimization of the dose.

7. The composition according to claim 1, wherein the composition provides a daily dose, which is the dose administered over a period of 24 hours, of between about 30 and about 110 mg.

8. The composition according to claim 7, wherein the dose is a nominal dose

9. The composition according to claim 1, wherein the composition allows doses to be administered at regular and frequent intervals providing maintenance therapy.

10. The composition according to claim 1, wherein the composition provides a Cₘₐₓ within less than about 10 minutes of administration by pulmonary inhalation.

11. The composition according to claim 1, wherein the composition provides a dose dependent Cₘₐₓ upon administration by pulmonary inhalation.

12. The composition according to claim 1, wherein the composition provides a therapeutic effect in about 10 minutes or less following administration by pulmonary inhalation.

13. The composition according to claim 1, wherein the composition comprises at least about 10% (by weight) apomorphine.

14. The composition according to claim 1, further comprising an additive material.

15. The composition according to claim 1, further comprising particles of an inert excipient material.

16. A blister or capsule containing a composition according to claim 1.

17. An inhaler device comprising a composition according to claim 1.

18. The inhaler device according to claim 17, wherein the device is a dry powder inhaler, a pressurized metered dose inhaler or a nebuliser.

19. A process for preparing a composition comprising:
providing apomorphine in dry powder form.

20. A method of treating a disease of the central nervous system comprising the steps of:
providing a subject having a disease of the central nervous system and
administering a composition according to claim 1 to the provided subject by pulmonary inhalation, thereby treating the disease of the central nervous system.
